(19) United States
(12) Patent Application Publication
(10) Pub. No.: US 2006/0122482 A1
(43) Pub. Date: Jun. 8, 2006

(54) Medical image acquisition system for receiving and transmitting medical images instantaneously and method of using the same

(75) Inventors: Mark Mariotti, Chelmsford, MA (US); John Melville, Boxford, MA (US); Joseph Muratore, Chelmsford, MA (US); Anthony Molinari, Grafton, MA (US)

Correspondence Address: Law Offices of Michael Leccese, P.C.;Michael Leccese, Esq., Suite 202, 790 Turnpike St., North Andover, MA, 01845, US

(73) Assignee: Foresight Imaging Inc., Lowell, MA (US)

(21) Appl. No.: 11/282,522

(22) Filed: Nov. 19, 2005

Related U.S. Application Data
(60) Provisional application No. 60/522,932, filed on Nov. 22, 2004.

(51) Int. Cl. A61B 5/05

(52) U.S. Cl. 600/407

(57) ABSTRACT

An apparatus and method of acquiring video data from a source, having a source transmission going to a home base computer, a remote computer or image/audio receiving device. The home base computer receiving, compressing, converting the acquired data from a video format to a digital image format, transmitting the data to a remote system in real time, querying a consulting physician, the consulting physician reviewing the real time data and communicating back to the home base computer via a voice conversation. The home base computer storing and archiving data. The home base computer having the capability of transmitting the video signal from medical imaging devices at their native resolutions using real time, instantaneously, compressing and transmitting over Internet protocol, IP, interfaces at diagnostic quality or near diagnostic quality. The home system transmitting a video stream in real time as well as a real time audio stream. Voice over IP transmissions of voice and modality audio from medical imaging devices. The present invention is also a collaborative voice conferencing system using real time audio streaming.

### FIELD

The invention relates generally to a medical image acquisition system and method of use, the acquisition system acquiring, compressing, and converting standard analog or digital video signals from medical equipment to digital images. The system adapted for receiving and transmitting still or motion medical images instantaneously along with audio steaming data.

### BACKGROUND

In the past, viewing captured medical image data was accomplished in less than real time, that is to say the image transmission has periods of sporadic or inconstant operation from the transmitting device to the receiving device. Present applications would require a user to wait for image generation, typically seconds and then possibly minutes for the image to become available at a central node for viewing. Real time or instantaneous describes an application which a computer program responds to stimuli within some small upper limit of response time (typically milliseconds).

The traditional way of capturing an image on a medical imaging machine commonly called a modality, generally consisted of an operator or technician first conducting a scan. Then using the modality to save the image, in still or video format, into the modality memory or into a main image storage database. Soon afterward perhaps downloading the image into a hospital database such as a PACS system, Picture Archiving and Communications System, for storage and later retrieval. The doctor would then access the PACS system to retrieve the image, the doctor at that time would call up the image, view and review the image and conceivably develop a diagnosis based on the information from the image. The advantage of the present invention accomplishes all these tasks in real time.

In order to provide quick and efficient medical care, information such as medical imaging must be presented to a consulting physician in faster time so the physician can make a timely diagnosis for the patient or make decisions about doing further imaging or possible therapy while the patient is still at the imaging station. In some cases the patient and physician may not be in the same location and the physician may find himself providing a diagnosis apart from the patient whether that physician is at another part of the hospital or at a remote location. The present invention allows a physician to view image data from any computer having a hospital LAN or Local Area Network connection or an Internet connection including a wireless network connection. A diagnosis or instruction function is provided in which a real time human conversation is taking place along with viewing of images from the modality. The system also allows for multi consultant availability and querying providing a multi-party conference capability.

Moreover many of the prior art devices do not supply an efficient cost effective solution to the problem of non real time image processing and communication, the inventors of this invention have filed a long felt need in the imaging arts.

### SUMMARY

This invention provides an apparatus and method of using and viewing real time instantaneously static and dynamic medical images, obtained from any medical imaging modality. The system can network real time images from one location to the next. Many times clinicians such as physicians have the need to view live and historic images to make proper diagnoses. More frequently the need arises for physicians to communicate images and voice data from remote locations for the purposes of making remote diagnoses and for training of staff in clinical settings, when the physician (or student staff) is not necessarily located in a traditional hospital setting.

It is an object of this invention to provide an apparatus and method of providing real time images along with audio communications in real time. The invention is directed to a device that can be used for the purpose of allowing an operator, mostly a technician of any medical imaging device to consult with a specialist, typically a doctor located anywhere a computer can be accessed. Medical imaging encompasses the full range of medical fields. Among the many medical imaging fields are ultrasound, CT/CAT scan or computerized tomography, fluoroscopy, endoscopy, MRI or magnetic resonance imaging, nuclear medicine, PET or positron emission tomography, echocardiorgaphy ultrasound, angiography and microscopy.

More particularly the invention uses present technology of today's computers, video auto-sensing frame capture electronic circuit boards, capture, compression and transmission software and point to point transmissions of the captured signal. In further detail the system applies existing digital technologies, acquiring an analog or digital video signal, transforming that signal into digital image data, compressing and transmitting the data over a computer network and using voice over internet protocol (IP), commonly known and used in the medical industry. Transmitting an image from one location to another over the network in real time, converting the image from a video format to a digital image format.

It is further object of this invention to frame grab images from selected medical devices, acquiring the image at the video rate of the modality and transmit those images at a speed such that the images can be viewed in real time, instantly. The speed of transmission can vary however depending on items such as processor speed, compression type, network bandwidth, and the complexity of the imagery. The acquisition software used in combination with the invention described herein makes up the system which acquires, compresses and sends the signal to the receiving device. Image transfer can be lossless or lossy, according to the user's settings. Multicasting, the ability to send data to more than one receiver at the same time, is possible from each video acquisition device (VAD), the system can provide access to all VAD's anywhere on the network.

It is a further object of this invention to include many features such as: non intrusive video capture and transmission for remote viewing, capture from any analog or digital video source, including s-video, composite color or monochrome, component RBG, DVI and HDMI. As well as the ability to preserve the original video source resolution for transmission to receiver locations, enabling a remote viewer to see the image at the same resolution as the original modality. The invention also has the ability to multicast video, modality audio and voice over existing data networks like local area networks (LAN) or wide area networks (WAN), the Internet and broadcast to multiple receiving units in the same network group. Point to point transmissions over existing data networks, broadcast to any selected Internet protocol (IP) address. In addition the systems can AVI record functionality for multicast and point to point. AVI is Audio/Video Interleaved, a computer movie file format. The system also has the ability to record the video and audio streams at high resolution. Also available is audio conferencing between multiple sites.

One illustration of this invention embodies a Streamview™ software module that can be integrated with any software application program as shown in the pictorial FIG. 5. that presents a live video window on a computer screen. Streamview™, has the capability of intercepting the video in that window and transmitting it to another software application program running on anotheromputer. The illustrated embodiment is comprised of two software application programs, one called TIMS™ (Tele-medicine Imagine Management System), the other called TIMS Consultant. TIMS is a server type program on a single computer that is acquiring the medical video signal and transmitting video and voice signals to a client program, TIMS Consultant is on a remote computer that is receiving the video and voice over IP transmission. Both programs use the StreamView module to manage the video and audio data streams. The TIMS computer is located with the medical imaging device or in a nearby facility. A remote consultant physician uses the TIMS Consultant application installed on any computer equipped with speakers, microphone and connected to a computer network. All communication, video and audio, is accomplished over the network. The TIMS Consultant computer can be located anywhere there is LAN or Internet connection. The system enables the Consultant, to view the images from the medical imaging system and discuss with the technician operating the medical device that is acquiring the images the best steps to take in the examining procedure. This allows for expert guidance to be given during the imagining session providing a potentially faster diagnosis and increasing the likelihood that the images taken are most useful. In this embodiment TIMS provides the real-time video and audio communication as well as a method of recording and transmitting images in DICOM format. DICOM, Digital Imagining and Communications in Medicine, is a medical imaging standard common in the medical industry. The embodiment can serve as the connection point between any medical imaging system and a hospital PACS, patient archival and communications system. The primary capability of the TIMS program is to connect older non-DICOM equipment to a hospital network, allowing imaging studies to be stored in PACS. It can also be used to connect DICOM compatible equipment if it desired to use some features contained in TIMS that are not available on the imaging system such as the TIMS Consultant feature.

It is further object of this invention to supply the necessary apparatus and method to send and receive medical images in real time as well as at the same time receive and send an audio stream. It is yet another object of this invention to provide and apparatus and method that is cost effective, simple, lightweight, long lasting and durable.

It is a further object of this invention to provide an apparatus and method that is simple by design and efficient in method and use.

The principles preferred embodiments and modes of operation of the present invention have been described in the forgoing specification. The invention which is intended to be protected herein, however, is not to be construed as limited to the particular embodiments disclosed, since these embodiments are to be regarded as illustrative rather than restrictive. Variations and changes may be made by others without departing from the spirit of this invention. Accordingly, it is expressly intended that all such variation and changes which fall within the spirit and scope of the claims be embraced thereby.

### BRIEF DESCRIPTION OF DRAWINGS

Other objects, features, and advantages will occur to those skilled in the art from the following description of an embodiment and the accompanying drawings, in which:

FIG. 1, shows a flow diagram of the system

FIG. 2, shows a typical transmitted image

FIG. 3, shows a typical software dialog box displayed on a computer screen wherein the operator can choose and page a consultant or consultants to consult with

FIG. 4, shows a typical software dialog displayed on a computer screen wherein the consultant can acknowledge a page

FIG. 5, shows a typical computer display screen listing consultants

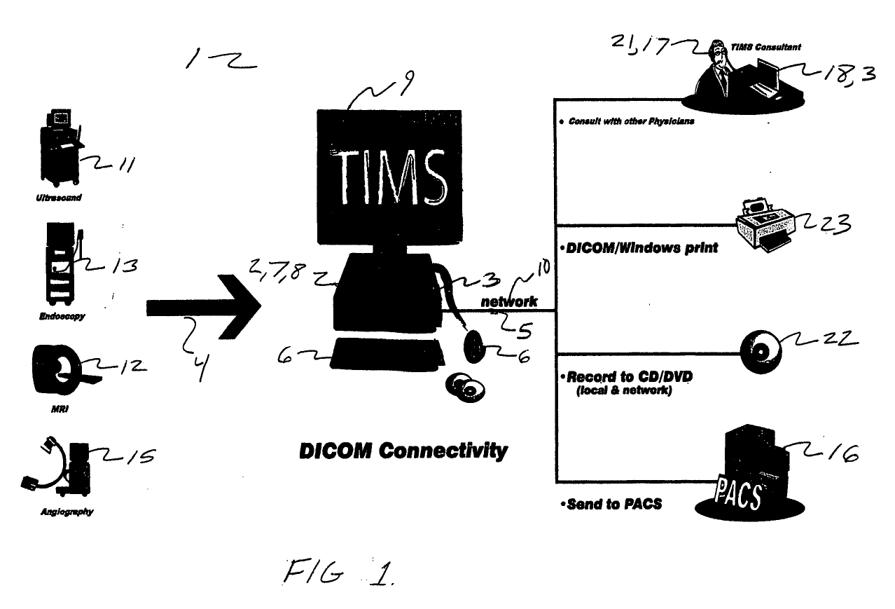

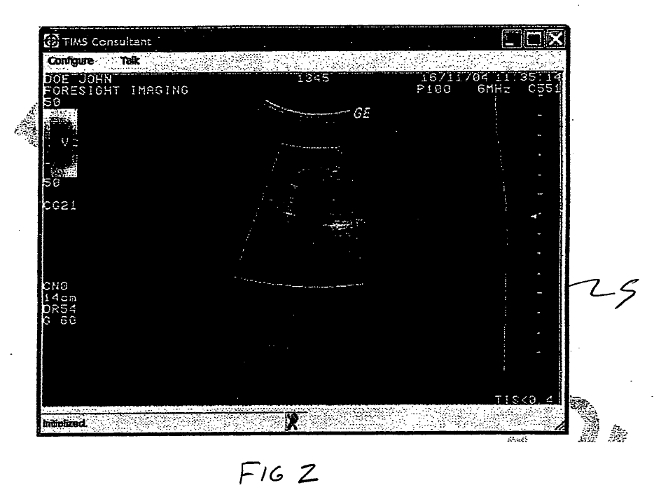

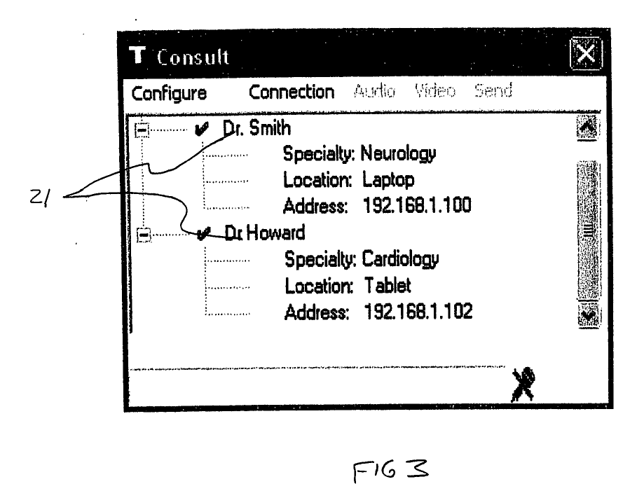

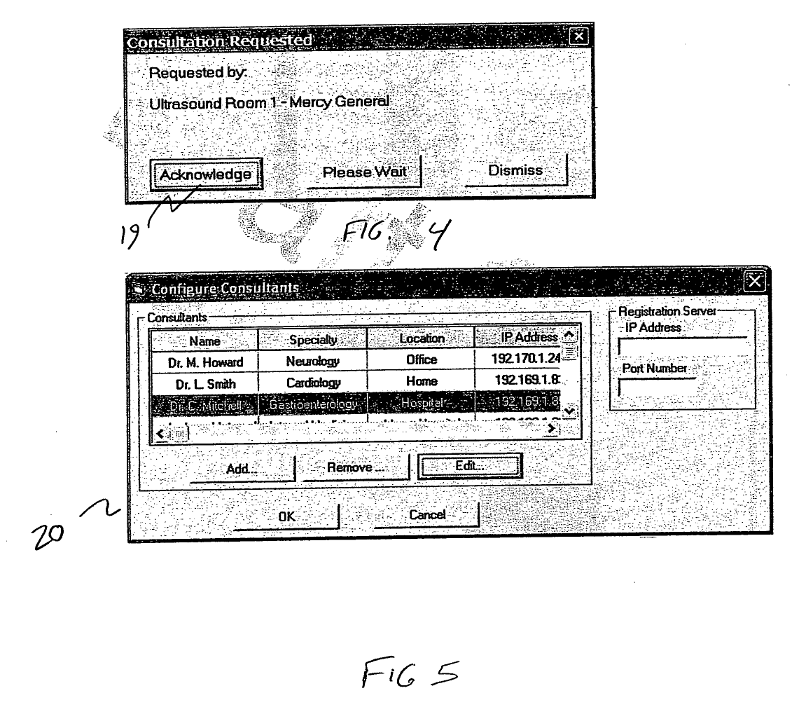

### DETAILED DESCRIPTION

A medical image acquisition system 1 and method of use adapted for receiving and transmitting medical images and audio signals coincidentally and instantaneously. A home base unit 2 which is typically located at a medical organization which includes a computer 3 having communications capability. The home 3 unit having the ability to receive 4 and send a plurality 5 of incoming and outgoing signals. The computer 2 commonly having input devices 6, a central processing unit 7, data storage device 8, a display 9, a network connection 10 and wireless communications provisions. In addition the system can also record 22 both image data signals as well as audio signals. The home base 2 unit is adapted for acquiring and transmission a plurality of medical imaging video signals as well as medical facility data base storage 16. In most cases the video signals are acquired from a medical device in that device's native resolutions, transmitting the signals instantaneously at their native resolutions to a remote device 18. Types of devices the image signals are received from include but not limited to, ultrasound 11, cat scan 12, fluoroscopy 13, endoscopy, magnetic imagining 15, nuclear medicine, echocardiogram ultrasound and microscopy. Converting the video signals to digital format and compressing for transmission.

Transmitting the signal to a remote-receiving device 5, commonly a computer. The home base receiving the medical imaging video signals, converting the signal to digital form, compressing the signal for transmission, transmitting the digital signals to the remote device. The home device connected to a network interconnection, typically a LAN or the Internet using standard IP, internet protocol. In some cases the communications between systems is wireless. The medical device 11, 12, 13, 15 or medical facility device 16 units video is connected to the home unit 2, the home unit then transmitting to the remote unit-receiving device. Typically a physician 17 or any selected person 21 located at the remote unit 18, 3 watching and directing the procedure. The home base 2 unit having instantaneous communications with the medical organization and remote unit. Transmitting of signals can be encrypted for secure transmissions. In most cases the medical image acquisition system is adapted for receiving the video image signal in a plurality of video signal types including, S-video, composite color or monochrome, component RGB [commonly know in the industry as the RGB color model that utilizes the additive model in which red, green, and blue light are combined in various ways to create other colors], DVI [commonly know in the industry as Digital Visual Interface a digital interface standard] and HDMI [High-Definition Multi-media Interface is an industry-supported, uncompressed, all-digital audio/video interface]. Also the medical image acquisition system unit includes a storage device 22 adapted for archiving 23 the video signal in a predetermined digital format including DICOM, AVI.

In one illustration the medical image acquisition system is communicating with the home base 2, collaborating, and conferencing many persons at the same time 21. For example the physician 18 can view the instant image and conference through audio in real time with the operator at the home base system. This can operate by the home base unit paging the remote unit including text messaging the remote unit, alerting the remote unit of a conferencing request. The system can consult with a plurality of consultants at the same time 20. The Physician then acknowledging 19 the request and the home unit subsequently transmits the images and consults with the doctor.

What is claimed is:

1. A medical image acquisition system and method adapted for receiving and transmitting medical images instantaneously, comprising;
a home base unit which is located at a medical organization which includes a computer having communications capability, wherein the home base unit is adapted for acquisition and transmission of a plurality of medical imaging video signals wherein the video signals are acquired from a transmitting medical device in the medical device's native resolutions, transmitting the signals instantaneously at their native resolutions to,
a remote receiving device, the home base receiving the medical imaging video signal, compressing the signal, converting the signal to digital form for transmission, transmitting the digital signals to the remote device, and;
a network interconnection to the home base unit, transmitting medical device unit and remote receiving device wherein the home base unit having instantaneous communications with the medical organization and remote unit.

2. A medical image acquisition system as in claim 1, wherein the home base acquires signals from a plurality of medical imaging systems including, ultrasound, cat scan, fluoroscopy, endoscopy, magnetic imagining, nuclear medicine, echocardiogram ultrasound and microscopy.

3. A medical image acquisition system as in claim 1, wherein the internet connection uses a standard internet protocol.

4. A medical image acquisition system as in claim 1, wherein with remote unit is a computer system.

5. A medical image acquisition system as in claim 1, wherein the home base unit is adapted for receiving the video image signal in a plurality of video sources including, S-video, composite color and monochrome, component RGB, DVI and HDMI.

6. A medical image acquisition system as in claim 1, wherein the home unit includes a storage device adapted for archiving the video signal in a predetermined digital format including Digital Imaging and Communications for Medicine, Audio/Video Interleaved.

7. A medical image acquisition system as in claim 1, wherein the communications includes wireless communications.

8. A medical image acquisition system as in claim 1, wherein transmitting data includes transmitting the data in secure encryption protocols.

9. A medical image acquisition system as in claim 1, wherein the transmitting video signal resolution is the same resolution as the received signal.

10. A medical image acquisition system as in claim 1, wherein the home unit receives a plurality of signals from a plurality of medical devices, converting and transmitting to a plurality of remote units.

11. A medical image acquisition system as in claim 1, wherein the medical image acquisition system is transmitting and receiving audio signals instantaneously.

12. A medical image acquisition system as in claim 1, wherein the medical image acquisition system is adapted for recording image video.

13. A medical image acquisition system as in claim 1, wherein the medical image acquisition system is adapted for recording audio signals.

14. A medical image acquisition system as in claim 1, wherein the remote location communicates with the home base, for the purpose of collaborating and conferencing.

15. A medical image acquisition system as in claim 1, wherein the home base unit pages the remote unit including text messaging the remote unit, alerting the remote unit of a conferencing request.

16. A medical image acquisition system as in claim 15, wherein the person at a remote computer hears the paging and acknowledges the page and then receiving transmitted image and audio signals.

17. A method for using a medical image medical image acquisition system for receiving and transmitting medical images instantaneously, comprising;
acquiring image data from an image producing medical device by receiving medical video signals into a computer, converting the video signals to digital images, compressing the images, transmitting consultant request signal to a remote system, receiving the consultant acknowledging the request, transmitting streaming image and audio data instantaneously to a remote communication system, the person at the remote system reviewing the image data, directing the user of the image producing medical device in instant audio, the medical acquisition system storing the image data, the medical acquisition system storing the audio data.

18. A method for using a medical image acquisition system for receiving and transmitting medical images instantaneously as in claim 17 wherein, transmitting to a consultant includes but not limited to a physician, nurse, and medical personal.
